(19) United States
(12) Patent Application Publication
(10) Pub. No.: US 2005/0182316 A1
(43) Pub. Date: Aug. 18, 2005

(54) Method and system for localizing a medical tool

(76) Inventors: Everette C. Burdette, Champaign, IL (US); Christopher Alix, Urbana, IL (US); Lippold Haken, Champaign, IL (US)

Correspondence Address: THOMPSON COBURN, LLP, ONE US BANK PLAZA, SUITE 3500, ST LOUIS, MO, 63101, US

(21) Appl. No.: 10/902,429

(22) Filed: Jul. 29, 2004

Related U.S. Application Data
(63) Continuation-in-part of application No. 10/230,986, filed on Aug. 29, 2002.
(60) Provisional application No. 60/491,634, filed on Jul. 30, 2003.

(51) Int. Cl. A61B 5/05

(52) U.S. Cl. 600/424

(57) ABSTRACT

A method of localizing a medical tool, the method comprising: (1) generating an image of a reference target with a camera that is attached to a medical tool, wherein the reference target is remote from the medical tool and located in a room at a known position relative to a coordinate system; and (2) determining the position of the medical tool relative to the coordinate system at least partially on the basis of the generated image of the reference target. Examples of medical tools that can be localized in accordance with the present invention include medical imaging devices, surgical instruments, and bite blocks.

### CROSS-REFERENCE TO RELATED APPLICATIONS

This application claims priority to provisional patent application Ser. No. 60/491,634 entitled “Method and System for Localizing a Medical Imaging Probe” filed Jul. 30, 2003, the entire disclosure of which is incorporated herein by reference.

This application is also a continuation-in-part of pending patent application Ser. No. 10/230,986 entitled “Method and Apparatus for Spatial Registration and Mapping of a Biopsy Needle During a Tissue Biopsy” filed Aug. 29, 2002, the entire disclosure of which is incorporated herein by reference.

### FIELD OF THE INVENTION

The present invention relates generally to the localization of a medical tools, particularly medical imaging devices. In particular, the present invention relates to the localization of a medical imaging probe in real-time as the probe is used in connection with generating a medical image of a patient.

### BACKGROUND OF THE INVENTION

With various medical procedures in which medical images of a patient are generated using a medical imaging device such as a medical imaging probe, it is often important to precisely know the position of content depicted in a medical image relative to a fixed coordinate system. This content depicts a region of interest (ROI) of the patient. Such a position determination allows for precise patient diagnoses, precise formulation of treatment plans, precise targeting of therapy treatments, and the like.

For example, when preparing an external beam radiation treatment plan for treating prostate cancer, it is highly important to target the radiation beam as closely as possible to diseased regions of the prostate to thereby minimize damage to nearby healthy tissue. In this case, the ROI is a diseased region of the prostate or the entire prostate with a minimized treatment margin surrounding the prostate. Once the diseased region is identified in the medical images of the prostate, the question becomes how to accurately target the radiation beam to the ROI and spare adjacent critical structures. To achieve such targeting, it is desirable to spatially register the medical images relative to the fixed coordinate system of the radiation beam source. In this process, the known and relatively constant variables are the position of the radiation beam relative to the fixed coordinate system, the position of the ROI relative to the probe's field of view, and the probe's field of view relative to the probe's position. The missing link in this process is the position of the medical imaging probe relative to a coordinate system such as the coordinate system of the radiation source at the time the probe obtains data from which the medical image of the patient is generated.

A variety of techniques, referred to generally as localization systems, are known in the art to determine the position of a medical imaging probe relative to a fixed coordinate system. Examples of known localization systems can be found in U.S. Pat. Nos. 5,383,454, 5,411,026, 5,622,187, 5,769,861, 5,851,183, 5,871,445, 5,891,034, 6,076,008, 6,236,875, 6,298,262, 6,325,758, 6,374,135, 6,424,856, 6,463,319, 6,490,467, and 6,491,699, the disclosures of all of which are incorporated herein by reference.

For example, it is known to mount the medical imaging probe in a positionally-encoded holder assembly, wherein the assembly is located at a known position in the coordinate system (and therefore the probe's position in the coordinate system is also known) and wherein the probe is moveable in known increments in the x, y, and/or z directions. However, because such localization systems require the use of a holder assembly, the probe's range and manner of movement is limited to what is allowed by the encoder rather than what is comfortable or most accurate for the medical professional and patient.

It is also known to mount a medical imaging probe in a holder assembly, wherein light sources such as light emitting diodes (LEDs) are affixed either to the probe itself or to the holder assembly, and wherein a camera is disposed elsewhere in the treatment room at a known position such that the LEDs are within the camera's field of view. Applying position determination algorithms to points in the camera images that correspond to the LEDs, the probe's position relative to the system's fixed coordinate system can be ascertained.

In connection with freehand medical imaging probes, similar localization systems are used wherein LEDs are affixed to the probe, wherein a camera that is disposed elsewhere in the treatment room at a known location is used to generate images of those LEDs, and wherein a position determination algorithm is used to process the camera images to localize the probe in 3D space.

However, because treatment rooms typically offer a limited variety of choices for camera placement locations, it is often the case that a close spatial relationship cannot be maintained between the camera and the LEDs it seeks to track. Thus, it is believed that these known camera-based localization systems suffer from potential line-of-sight (LOS) problems as people in the treatment room move about or as the probe is moved about during the imaging process. These same problems are believed to exist in connection with localizing medical tools other than medical imaging devices (such as surgical instruments).

### SUMMARY OF THE INVENTION

In view of these and other opportunities for improvement in conventional localization systems, the inventors herein have developed the present invention. As a unique and elegantly simple improvement to the prior art discussed above, the inventors herein have, in their preferred embodiment, attached a tracking camera to a medical imaging probe and placed the reference target tracked by the camera elsewhere in the treatment room at a known location. Because there are a much greater number of options for reference target placement in a treatment room than there are for camera placement due to the reference target's small size and easy maneuverability, the present invention allows for a close spatial relationship to be maintained between the tracking camera and the reference target, thereby minimizing the risk for LOS problems. Further, the configuration of the present invention can provide improved accuracy at lower cost by avoiding the long distances that are usually present between the LEDs and room-mounted cameras of conventional systems.

According to one aspect of the invention, disclosed herein is a method of localizing a medical tool, the method comprising: (1) generating an image of a reference target with a camera that is attached to a medical tool, wherein the reference target is remote from the medical tool and located in a room at a known position relative to a coordinate system; and (2) determining the position of the medical tool relative to the coordinate system at least partially on the basis of the generated image of the reference target.

In preferred embodiments, the medical tool can be a medical imaging device (such as a freehand ultrasound probe), a surgical instrument, or a bite block, as described in greater detail below.

Also disclosed herein is a system for localizing a medical imaging device, the system comprising: (1) a reference target having a known position in a fixed coordinate system; (2) a medical imaging device having a field of view and being configured to receive data from which a medical image of a patient is generated, the medical imaging device being remote from the reference target; (3) a camera attached to the medical imaging device for tracking the reference target and generating at least one image within which the reference target is depicted; and (4) a computer configured to (a) receive the camera image and (b) process the received camera image to determine the position of the medical imaging device's field of view relative to the coordinate system.

Also disclosed herein is a system for localizing a medical tool, the system comprising: (1) a medical tool for use in a medical procedure with a patient; (2) a localization system associated with the medical tool that locates the medical tool in a three-dimensional coordinate system, the localization system comprising a reference target having a fixed and known position in the coordinate system, the reference target being remote from the medical tool; and (3) a computer in communication with the localization system, the computer being programmed to (a) receive data from the localization system, and (b) determine the position of the medical tool in the coordinate system at least partially on the basis of data received from the localization system.

According to another aspect of the present invention, disclosed herein is a medical tool having a tracking camera attached thereto in a known spatial relationship with respect to a point of interest on the medical tool. The camera is preferably attached to the tool such that the camera images a reference target remote from the medical tool while the medical tool is being used as part of a medical procedure, and wherein the reference target is disposed in the same room as the medical tool at a known position in the room relative to a 3D coordinate system.

According to yet another aspect of the present invention, disclosed herein is a computer programmed with executable instructions to process camera images received from a medical tool-mounted tracking camera together with known position variables to determine the position of the medical tool relative to the coordinate system.

In a preferred embodiment wherein the medical tool is an imaging probe, the tracking camera is attached to the imaging probe at a known position and orientation with respect to the imaging probe's field of view. Further, the reference target is located in the treatment room at a known position in the coordinate system and within the field of view of the tracking camera as the probe is put to use. The reference target includes a plurality of markings that are identifiable within the camera images, wherein the markings have a known spatial relationship with each other. On the basis of these known variables, a computer programmed with a position determination algorithm can process images from the tracking camera in which the reference target markings are identifiable to determine the position of the probe relative to the coordinate system. As a result of determining the probe's positioning relative to the coordinate system, medical images generated through the use of the probe can be spatially registered to that same coordinate system.

The localization technique of the present invention is suitable for use with any medical procedure in which spatially registered medical images or accurate localized treatments are useful, including but not limited to the planning and/or targeting of spatially localized therapy (e.g., spatially localized drug delivery, spatially localized radiotherapy including but not limited to external beam radiation therapy treatment planning, external beam radiation treatment delivery, brachytherapy treatment planning, brachytherapy treatment delivery, etc.), pre-biopsy planning, and biopsy execution.

The preferred imaging modality for use with techniques of the present invention is ultrasound. However, it should be noted that other imaging modalities may also be used, including but not limited to imaging modalities such as x-ray, computed tomography (CT), cone-beam CT, and magnetic resonance (MR).

These and other features and advantages of the present invention will be in part pointed out and in part apparent upon review of the following description and the attached figures.

### BRIEF DESCRIPTION OF THE DRAWINGS

FIG. 1 is a block diagram overview of a preferred embodiment of the localization system of the present invention, wherein a transrectal ultrasound probe is localized;

FIG. 2 is a block diagram overview of a preferred embodiment wherein the localization system uses a transabdominal ultrasound probe;

FIG. 3 is a depiction of the preferred embodiment wherein the localization system uses a transabdominal ultrasound probe;

FIG. 4 illustrates a preferred reference target pattern;

FIG. 5 illustrates an exemplary localizable surgical instrument in accordance with the localization technique of the present invention; and

FIG. 6 illustrates an exemplary localizable bite block in accordance with the localization technique of the present invention.

### DETAILED DESCRIPTION OF THE PREFERRED EMBODIMENT

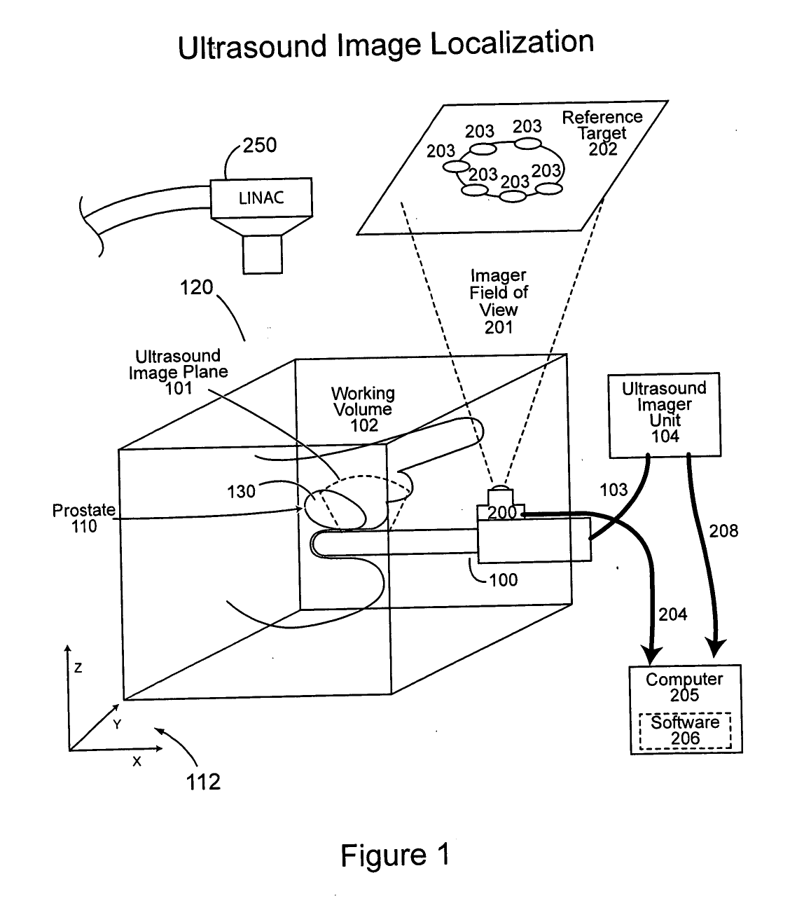

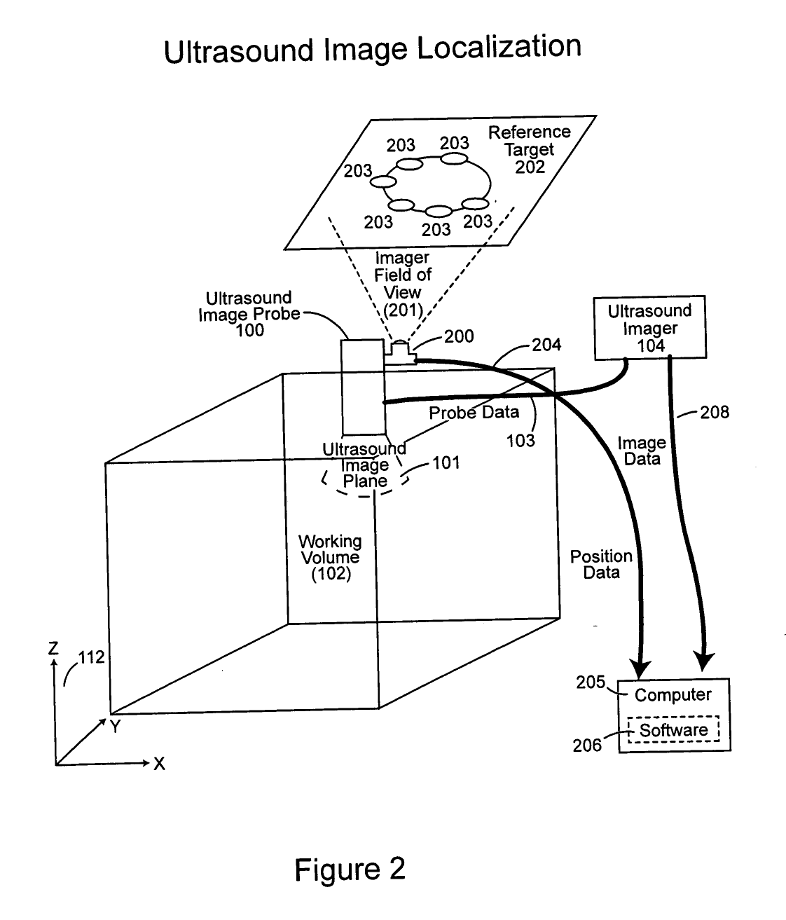

FIG. 1 illustrates an overview of a preferred embodiment of the localization system of the present invention, as applied to prostate treatment via an external beam radiation therapy procedure. Commonly-owned pending application Ser. No. 10/286,368, filed Nov. 1, 2002, (the entire disclosure of which is incorporated herein by reference) discloses an exemplary system for external beam radiation therapy for which the present localization system is well suited. In FIG. 1, a linear accelerator (LINAC) 250 serves as a source of radiation beam energy for treating prostate lesions. Because of the present invention's probe localization, this beam of energy can be precisely targeted to diseased regions of the prostate 110. However, as noted above, the localization system is also highly suitable for use with other medical procedures. Further, the target of medical imaging for the present invention need not be limited to a patient's prostate. Although spatial registration for medical images of a patient's prostate represents a unique and highly useful application of the present invention given the considerations involved with prostate treatment due to daily movement of the prostate within the patient, the medical imaging target of the present invention can be any soft tissue site of a patient's body including but not limited to the pancreas, kidney, bladder, liver, lung, colon, rectum, uterus, breast, head, neck, etc. Most internal organs or soft tissue tumors that move to some degree within the patient would be candidates for targeting using the localization approach of the present invention.

In FIG. 1, a target volume 110 (or ROI) is located within a working volume 102. For the example of FIG. 1, the target volume 110 would be a patient's prostate or a portion thereof, and the working volume 102 would be the patient's pelvic area, which includes sensitive tissues such as the patient's rectum, urethra, and bladder. Working volume 102 is preferably a region somewhat larger than the prostate, centered on an arbitrary point on a known coordinate system 112 where the prostate is expected to be centered during the external beam radiation therapy procedure.

A medical imaging device 100, in conjunction with an imaging unit 104, is used to generate medical image data 206 corresponding to objects within the device 100's field of view 101. The device 100 may be a phased array of transducers, a scanned transducer, receiver, or any other type of known medical imaging device, either invasive or non-invasive. During a planning session or treatment session for external beam radiation therapy, the target volume 110 will be within the imaging device's field of view 101. Preferably, the medical imaging device 100 is an ultrasound probe and the imaging unit 104 is an ultrasound imaging unit. Even more preferably, the ultrasound probe 100 is a transabdominal or linear array imaging probe, a breast imaging probe, a transrectal ultrasound probe, or an intracavity ultrasound probe. Together, the ultrasound probe 100 and ultrasound imaging unit 104 generate a series of spaced two-dimensional images (slices) of the tissue within the probe's field of view 101. Although ultrasound imaging is the preferred imaging modality, as noted above, other forms of imaging that are registrable to the anatomy may be used in the practice of the present invention.

Also, in the example of FIG. 1, the imaging probe 100 is a freehand imaging probe. It is believed that the present invention is particularly valuable for use in connection with localizing freehand probes because, while freehand probes provide medical practitioners with unparalleled maneuverability during imaging, they also present difficulties when it comes to localization because of that maneuverability. However, given the present invention's localization abilities, a medical practitioner's freedom to maneuver the imaging probe is not hindered by the constraints inherent to conventional localization techniques. It is worth noting though, that in addition to localizing freehand probes, the present invention can also be used to localize non-freehand probes such as probes that are disposed in a holder assembly or articulable arm of some kind.

It is important that the exact position and orientation of ultrasound probe 100 and its field of view 101 relative to the known three-dimensional coordinate system 112 be determined. A preferred point of reference for the coordinate system, in external beam radiation therapy applications, is the machine isocenter of the LINAC (Linear Accelerator) 250. This isocenter is the single point in space about which the LINAC gantry and radiation beam rotates. To localize the ultrasound probe to the coordinate system 112, the localization technique of the present invention is used.

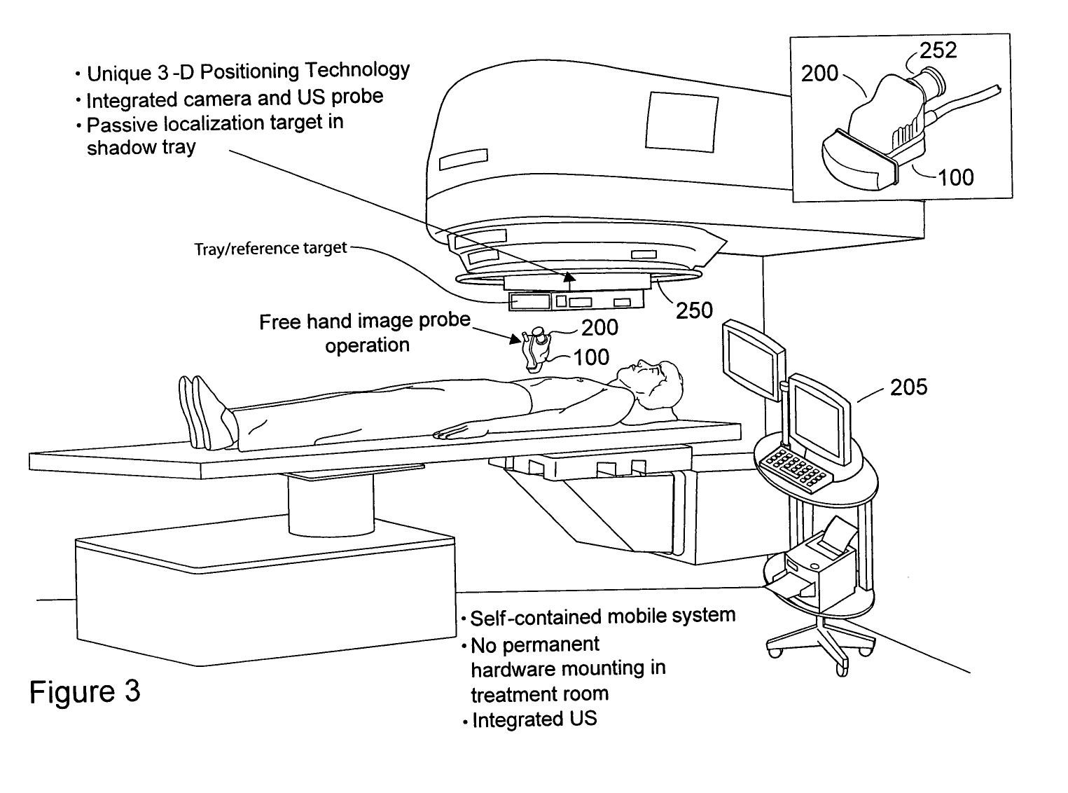

This localization technique uses a frameless stereotactic system wherein a tracking camera 200 is attached to the ultrasound probe 100 at a known position and orientation relative to a point of interest on the probe (preferably, within probe's field of view 101). When it is said that the tracking camera is “attached” to the ultrasound probe, it should be understood that this would include disposing the tracking camera on the probe directly via a single enclosure combining the two, disposing the tracking camera on the probe through a collar around the probe, wherein the tracking camera is directly affixed to the collar via a clamshell-like device, attaching the camera to the probe directly with a clamp. As would be understood by those of ordinary skill in the art, any of a number of known techniques can be used to appropriately attach the camera to the probe. Further still, the tracking camera 200 may also be detachable from the probe, although this need not be the case. The preferred attachment method is to incorporate a single housing that encompasses the camera 200 (except for the camera lens 252) and the probe 100 (except for the active transducer coupling window region), as shown in FIG. 3.

Various camera devices may be used in the practice of the present invention including but not limited to a CCD imager, a CMOS sensor type camera, and a non-linear optic device such as a camera having a fish-eye lens (which allows for an adjustment of the camera field of view 201 to accommodate volumes 102 of various sizes). In general, a negative correlation is expected between an increased size of volume 102 and the accuracy of the spatial registration system. Also, tracking camera 200 preferably communicates its image data 204 with computer 205 as per the IEEE-1394 standard.

Camera 200 is preferably mounted at a position and orientation on the probe 100 that minimizes reference target occlusion caused by the introduction of foreign objects (for example, the physician's hand, surgical instruments, portions of the patient's anatomy, etc.) in the camera field of view 201. Further, it is preferred that the camera 200 be mounted on the probe 100 as close as possible to the probe's field of view (while still keeping reference target 202 within camera field of view 201) because any positional and orientation errors with respect to the spatial relationship between the camera and probe field of view are magnified by the distance between the camera and probe field of view. A preferred location of the camera attachment to the probe matches the location of the hand grip for manipulation of the probe. The camera lens views above the hand grip toward the reference target and the imaging probe field of view is below the hand grip and probe.

A reference target 202 is disposed at some location, preferably fixed and preferably above or below the patient examination table, in the room 120 that is within the camera 200's field of view 201 and known with respect to the coordinate system 112. Preferably, reference target 202 is positioned such that, when the probe's field of view 101 encompasses the target volume 110, reference target 202 is within camera field of view 201. For external beam radiation therapy of the abdominal region, one preferred location of the reference target 202 is in the shadow tray or blocking tray of the LINAC. The block tray in some LINAC configurations inserts into the wedge tray slot. Another preferred location is in the wedge tray of the LINAC. The wedge tray in most LINAC configurations is located immediately on the treatment head of the LINAC gantry. The reference target can be placed in the selected tray slot of the LINAC and used to localize the targeting system, and then removed from the tray just prior to delivering the radiation treatment.

Reference target 202 is preferably a planar surface supported by some type of floor-mounted, table-mounted, or ceiling-mounted structure. Further, reference target 202 includes a plurality of identifiable marks 203 thereon, known as fiducials. Marks 203 are arranged on the reference target 202 in a known spatial relationship with each other.

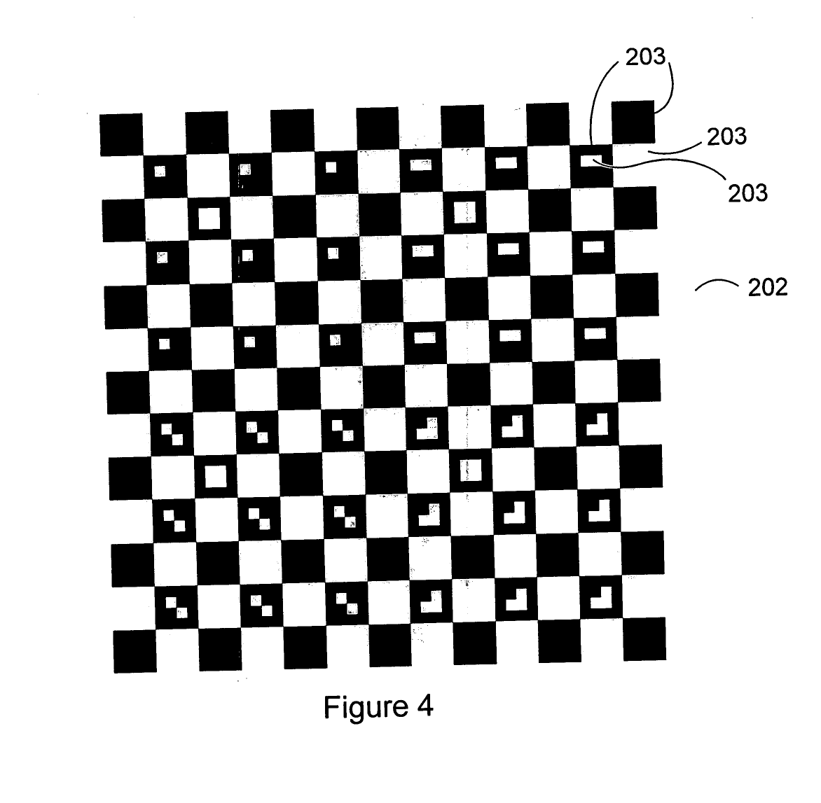

The identifiable marks 203 are preferably passive reflectors or printed marks visible to the camera 200 such as the intersection of lines on a grid, the black squares of a checkerboard, or some other pattern of markings on the room's wall or ceiling. FIG. 4 depicts a preferred checkerboard pattern for the reference target 202, wherein some of the checkerboard marks 203 include further geometric shapes and patterns.

However, other types of fiducials may be used such as light emitting diodes (LED's) or other emitters of visible or infrared light to which the camera 200 is sensitive. Any identifiable marks 203 that are detectable by the camera 200 may be used provided they are disposed in a known spatial relationship with each other. Further still, the camera can be replaced by an electromagnetic sensor or acoustic sensor, and the reference target replaced with electromagnetic emitters or acoustic emitters.

It is advantageous for the marks 203 to be arranged in a geometric orientation, such as around the perimeter of a rectangle or the circumference of a circle. Such an arrangement allows computer software 206 to apply known shape-fitting algorithms that filter out erroneously detected points to thereby increase the quality of data provided to the position-determination algorithms. Further, it is preferable to arrange the marks 203 asymmetrically with respect to each other to thereby simplify the process of identifying specific marks 203. For example, the marks 203 may be unevenly spaced along three sides of a rectangle or along a circular arc.

The number of marks 203 needed for the reference target is a constraint of the particular position-determination algorithm selected by a practitioner of the present invention. Typically a minimum of three marks 203 are used. In the preferred embodiment of FIG. 4, a checkerboard pattern with numerous marks 203 is used. In general, the positional and orientational accuracy of the localization system increases as redundant marks 203 are added to the reference target 202. Such redundant marks 203 also help minimize the impact of occlusion. The size of the marks 203 is unimportant provided they are of sufficient size for their position within the camera image to be reliably determined.

To calibrate the tracking camera 200 to its surroundings, the camera 200 is placed at one or more known positions relative to the coordinate system 112. In one preferred embodiment, the known positions of the camera relative to the target in the coordinate system are determined by precisioned machined mounting positions of exact known location in a metal plate into which the camera is inserted. When the camera 200 is used to generate an image of the reference target 202 from such known positions, the images generated thereby are to be provided to computer 205. The positions provide for placing the camera at various orientations which are communicated to the software. Software 206 that is executed by computer 205 includes a module programmed with executable instructions to identify the positions of the marks 203 in the image. The software 206 then applies a position-determination algorithm to determine the position and orientation of the camera 200 relative to the reference target 202 using, among other things, the known camera calibration positions, as is known in the art. Once the position and orientation of the camera 200 relative to the reference target 202 are known from one or more positions and at one or more orientations within the coordinate system 112, the computer 205 has calibration data that allows it to localize the position and orientation of the camera at a later time relative to the coordinate system 112. Such calibration can be performed regardless of whether the camera 200 is disposed on the probe 100. It may also be performed with the camera 200 disposed on the probe 100. The working volume is determined by the size of the region of the field of view of the camera relative to the visibility of the active sources or passive targets.

After calibration has been performed, the ultrasound probe 100 (with camera 200 attached thereto at a known position and orientation relative to the probe's field of view 101) can be used in “freehand” fashion with its location determined by computer 205 so long as the reference target 202 remains in the camera field of view 201. When subsequent camera image data 204 is passed to computer 205 via any known connection such as Firewire (IEEE 1394), Camera Link, or other suitable methods, software 206 (which may be instructions stored in the computer's memory, hard drive, disk drive, on a server accessible by the computer 205, or in other similar manner) applies similar position-determination algorithms to determine the position and orientation of the camera 200 relative to the reference target 202. By derivation, software 206 is then able to (1) determine the position and orientation of the camera 200 relative to the coordinate system 112 (because the position of the reference target 202 in coordinate system 112 is known), (2) determine the position and orientation of the probe field of view 110 relative to the coordinate system 112 (because the position and orientation of the camera 202 relative to the probe field of view 101 is known and because, as stated, the position and orientation of the camera 200 relative to the coordinate system 112 has been determined), and (3) determine the position and orientation of the content of the ultrasound image produced by the ultrasound probe 100 relative to the coordinate system 112 (because the ultrasound image contents have a determinable spatial relationship with each other within the probe's field of view 101 and because the relationship between the coordinate system and the camera are determinable based upon the camera calibration and the known relationship between the target and the coordinate system).

Position-determination algorithms are well-known in the art. Examples are described in Tsai, Roger Y., “An Efficient And Accurate Camera Calibration Technique for 3D Machine Vision”, Proceedings of IEEE Conference on Computer Vision and Pattern Recognition, Miami Beach, Fla., 1986, pages 364-74 and Tsai, Roger Y., “A Versatile Camera Calibration Technique for High-Accuracy 3D Machine Vision Metrology Using Off-the Shelf TV Cameras and Lenses”, IEEE Journal on Robotics and Automation, Vol. RA-3, No. 4, August 1987, pages 323-344, the entire disclosures of which are incorporated herein by reference. A preferred position-determination algorithm is an edge-detection, sharpening and pattern recognition algorithm that is applied to the camera image to locate and identify specific marks 203 on the target 202 with subpixel accuracy. The algorithm uses information from the camera image to locate the edges or corners of the reference target objects in space relative to each other and between light and dark areas. Repeated linear minimization is applied to the calculated location of each identified mark 203 in camera image coordinates, the known location of each identified point in world coordinates, vectors describing the location and orientation of the camera in world coordinates, and various other terms representing intrinsic parameters of the camera. The position and orientation of the ultrasound image is computed from the position and orientation of the camera and the known geometry of the probe/camera system.

One embodiment of the reference target may include sub-regions with additional patterns that are different in each sub-region. The software uses pattern recognition to analyze the presence and type of each sub-region pattern to determine which portion of the reference target is being viewed by the camera whenever the entire target is not visible to the camera. This information is used to extend the useful operational area or volume for localization of an image or surgical instrument.

Thus, as the ultrasound probe 100 is used to image the target volume 110 while the camera 200 tracks the reference target 202, camera image data 204 is provided to computer 205 and ultrasound image data 103 is provided to the ultrasound imaging unit 104 via a connection such as a coaxial cable. Software 206 executed by the computer operates to process the camera images received from the tracking camera 200 to localize the probe 100 through the above-described position determination algorithm. Once the probe 100 has been localized, the computer can also spatially register the ultrasound images 208 received via a connection such as a digital interface like Firewire or analog video from the ultrasound imager unit 104 through image registration techniques known in the art. This process is capable of occurring in real-time as the ultrasound sound probe is used to continuously generate ultrasound image data.

As mentioned above, in addition to the localization of medical imaging devices, the techniques of the present invention can also be applied to the localization of medical tools such as surgical instruments, bite blocks, and the like.

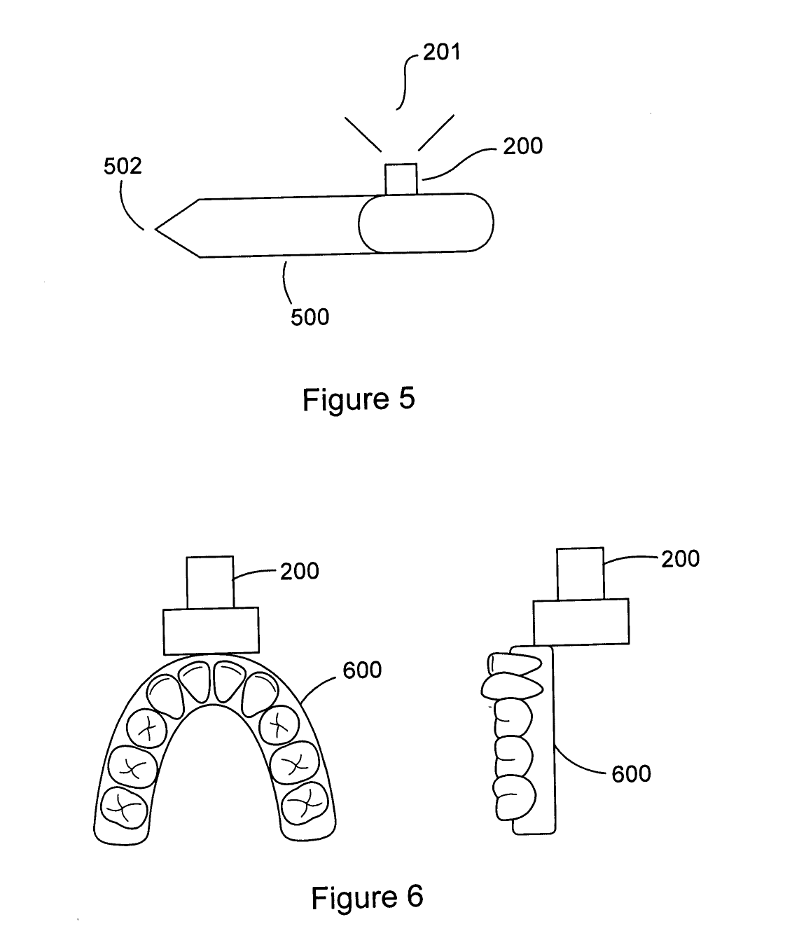

FIG. 5 depicts an example of a localizable surgical instrument 500. In FIG. 5, the surgical instrument 500, which may be a biopsy needle, a needle for delivery of therapeutic agent such as a drug, antibody, or biologic therapy, a thermal ablator, a cryosurgery probe, a cutting/cautery probe, or the like, includes a camera 200 attached at a position thereon having a known spatial relationship with respect to a point of interest 502 for the instrument 500. In this example, wherein the surgical instrument 500 is a needle used to deliver a therapeutic agent, the point of interest 502 is the needle end tip. However, the point of interest 502 for surgical instrument 500 need not be limited to needle tips; the point of interest may also include, depending on the surgical instrument, the distal end of a scalpel, or the distal portion of the active region of an ablator probe or cryotherapy probe. Localization of the surgical instrument 500 will proceed in accordance with the techniques described in connection with medical imaging devices, thus allowing a surgeon to accurately determine, in real-time, the location of point of interest 502 in a known 3D coordinate system.

FIG. 6 depicts an example (a top view and a side view) of a localizable bite block 600. A bite block 600 is a medical tool that is well-known in the art, particularly with respect to radiotherapy treatments of head or neck lesions/tumors, and is molded to fit a patient's teeth, preferably the patient's upper teeth. In FIG. 6, the bite block 600 includes a camera 200 attached at a position thereon having a known spatial relationship with respect to a treatment point on the patient's head or neck. Because the bite block is molded to fit the patient's teeth, which are a relatively stable reference point, the measurements that are made to determine the position of any head or neck lesions/tumors relative to a point on the bite block will be relatively constant from session to session. Through the use of the camera 200 in accordance with the teachings of the present invention, the bite block can also be localized relative to a fixed 3D coordinate system, thereby allowing the location of the lesion/tumor on the head or neck to also be accurately localized in the fixed 3D coordinate system based on the known relationship between the camera 200 and the lesion/tumor established from a CT scan of the patient with the bite block in place.

In both of the examples of FIGS. 5 and 6, the camera 200 is preferably attached to the medical tool such that the camera 200 is able to track a remote reference target while that tool is being used in connection with a medical procedure. In other words, during use of the medical tool, the camera 200 is positioned on the medical tool such that the reference target remains in the camera's field of view.

With the localization system of the present invention, and relative to conventional camera-based localization systems, the risk of occlusion is minimized through a greater likelihood of finding a location for the reference target that is within the camera's field of view.

While the present invention has been described above in relation to its preferred embodiment, various modifications may be made thereto that still fall within the invention's scope, as would be recognized by those of ordinary skill in the art following the teachings herein. As such, the full scope of the present invention is to be defined solely by the appended claims and their legal equivalents.

1. A method of localizing a medical imaging probe, the method comprising:
generating an image of a reference target with a camera that is attached to a medical imaging probe, wherein the reference target is remote from the probe and located in a room at a known position relative to a coordinate system; and
determining the position of the probe relative to the coordinate system at least partially on the basis of the generated image of the reference target.

2. The method of claim 1 wherein the determining step comprises determining, in substantially real-time, the position of the probe relative to the coordinate system at least partially on the basis of the generated image of the reference target.

3. The method of claim 2 wherein the reference target comprises at least one selected from the group consisting of a plurality of passive reflectors and a plurality of printed marks visible to the camera.

4. The method of claim 3 wherein the reference target comprises a plurality of printed marks arranged in a grid pattern.

5. The method of claim 4 wherein the grid pattern is a checkerboard pattern.

6. The method of claim 1 wherein the medical imaging probe is an ultrasound probe.

7. The method of claim 6 wherein the ultrasound probe is a freehand ultrasound probe.

8. The method of claim 6 further comprising:
generating at least one image of a patient's region-of-interest (ROI) through use of the ultrasound probe; and
spatially registering the at least one generated ROI image relative to the coordinate system at least partially on the basis of the determined probe position.

9. The method of claim 8 wherein the ROI is a patient's prostate.

10. A method of localizing a medical tool, the method comprising:
generating an image of a reference target with a camera that is attached to a medical tool, wherein the reference target is remote from the medical tool and located in a room at a known position relative to a coordinate system; and
determining the position of the medical tool relative to the coordinate system at least partially on the basis of the generated image of the reference target.

11. The method of claim 10 wherein the reference target comprises a plurality of identifiable marks thereon that are arranged in a known spatial relationship with respect to each other, and wherein the camera is attached to the medical tool at a position thereon having a known spatial relationship with respect to a point of interest on the medical tool.

12. The method of claim 11 wherein the medical tool is a medical imaging device.

13. The method of claim 12 wherein the medical imaging device is a freehand ultrasound probe.

14. The method of claim 11 wherein the medical tool is a surgical instrument.

15. The method of claim 11 wherein the medical tool is a bite block for use in treating head or neck lesions.

16. The method of claim 11 wherein the reference target is located in the room at a fixed position, and wherein the determining step comprises determining, in substantially real-time, the position of the medical tool relative to the coordinate system at least partially on the basis of the generated image of the reference target.

17. A system for localizing a medical imaging device, the system comprising:
a reference target having a known position in a fixed coordinate system;
a medical imaging device having a field of view and being configured to receive data from which a medical image of a patient within the device's field of view is generated, the medical imaging device being remote from the reference target;
a camera attached to the medical imaging device for tracking the reference target and generating at least one image within which the reference target is depicted; and
a computer configured to (a) receive the camera image and (b) process the received camera image to determine the position of the medical imaging device's field of view relative to the coordinate system in 3D coordinate space.

18. The system of claim 17 wherein the position of the medical imaging device's field of view relative to the position of the camera on the medical imaging device is known and wherein the computer is further configured to (a) determine, in substantially real-time, the position of the camera in the coordinate system at least partially on the basis of the received camera image, and (b) determine, in substantially real-time, the position of the medical imaging device's field of view at least partially on the basis of the determined camera position and the known position of the medical imaging device's field of view relative to the position of the camera on the medical imaging device.

19. The system of claim 17 wherein the reference target comprises a plurality of identifiable marks thereon that are arranged in a known spatial relationship with respect to each other.

20. The system of claim 19 wherein the reference target comprises a plurality of passive reflectors.

21. The system of claim 19 wherein the reference target comprises a plurality of printed marks visible to the camera.

22. The system of claim 21 wherein the printed marks are arranged in a grid pattern.

23. The system of claim 21 wherein the grid pattern is a checkerboard pattern.

24. The system of claim 17 wherein the medical imaging device is a freehand ultrasound probe.

25. The system of claim 24 wherein the camera is attached to the freehand ultrasound probe in close proximity to the probe's field of view.

26. The system of claim 24 wherein the freehand ultrasound probe is used to generate ultrasound images of an internal organ of a patient.

27. The system of claim 26 wherein the internal organ is the prostate.

28. The system of claim 26 wherein the origin of the fixed coordinate system is a machine isocenter of a linear accelerator (LINAC).

29. The system of claim 28 further comprising a LINAC for targeting a beam of radiation to a tumor on the patient's internal organ, wherein the LINAC includes a gantry, and wherein the reference target is located at a known position relative to the fixed coordinate system on the LINAC gantry.

30. The system of claim 28 further comprising a LINAC for targeting a beam of radiation to a tumor on the patient's internal organ, wherein the LINAC includes a tray, and wherein the reference target is located at a known position relative to the fixed coordinate system on the LINAC tray.

31. The system of claim 30 wherein the LINAC tray is a blocking tray.

32. The system of claim 30 wherein the LINAC tray is a wedge tray.

33. The system of claim 17 wherein the system comprises a system for localizing a medical imaging device used in connection with a tissue biopsy procedure.

34. The system of claim 17 wherein the system comprises a system for localizing a medical imaging device used in connection with a spatially localized drug delivery procedure.

35. The system of claim 17 wherein the system comprises a system for localizing a medical imaging device used in connection with a spatially localized radiotherapy procedure.

36. The system of claim 17 wherein the system comprises a system for localizing a medical imaging device used in connection with an external beam radiation therapy treatment planning session.

37. The system of claim 17 wherein the system comprises a system for localizing a medical imaging device used in connection with an external beam radiation therapy treatment delivery session.

38. A localizable medical imaging probe comprising:
a medical imaging probe having a field of view; and
a tracking camera attached to the medical imaging probe in a known spatial relationship with respect to the probe's field of view.

39. The probe of claim 38 wherein the tracking camera is attached to the probe such that the tracking camera images a reference target remote from the probe while the probe is being used to generate images of a patient, the reference target having a known position in a three-dimensional coordinate system.

40. The probe of claim 39 wherein the medical imaging probe is a freehand ultrasound probe.

41. The probe of claim 40 wherein the reference target is located at a fixed position in the coordinate system.

42. The probe of claim 40 wherein the tracking camera is a CCD imager.

43. The probe of claim 40 wherein the tracking camera is detachable from the probe.

44. A localizable medical tool comprising:
a medical tool for use in a medical procedure with a patient; and
a tracking camera attached to the medical tool in a known spatial relationship with respect to a point of interest associated with the medical tool.

45. The localizable medical tool of claim 44 wherein the tracking camera is attached to the medical tool such that the tracking camera images a reference target remote from the probe while the medical tool is being used in connection with a medical procedure, the reference target having a known position in a three-dimensional coordinate system.

46. The localizable medical tool of claim 45 wherein the tracking camera is a CCD imager.

47. The localizable medical tool of claim 45 wherein the medical tool is a medical imaging device.

48. The localizable medical tool of claim 45 wherein the medical tool is a surgical instrument.

49. The localizable medical tool of claim 45 wherein the medical tool is a bite block for use in treating head or neck lesions.

50. A computer readable medium for localizing a medical tool relative to a fixed coordinate system of a room, wherein the medical tool comprises a camera attached thereto at a known position relative to a point of interest on the medical tool, the camera being configured to image a reference target disposed in the room remotely from the medical tool, the reference target having a known position relative to the coordinate system, the computer readable medium comprising:
a plurality of executable instructions for processing camera images received from the camera together with known position data to determine the position of the medical tool relative to the coordinate system, wherein the camera images at least partially depict the reference target.

51. The computer readable medium of claim 50 wherein the medical tool is a medical imaging device.

52. The computer readable medium of claim 51 wherein the medical imaging device is a freehand ultrasound probe, wherein the reference target comprises a plurality of printed marks arranged in a grid pattern that are visible to the tracking camera, and wherein the coordinate system origin is the machine isocenter of a linear accelerator, and wherein the plurality of executable instructions further comprise a plurality of executable instructions for processing camera images received from the camera together with known position data to determine, in substantially real-time, the position of the medical tool relative to the coordinate system, wherein the camera images at least partially depict the reference target.

53. The computer readable medium of claim 52 wherein the plurality of executable instructions for processing camera images include executable instructions for applying an edge detection, sharpening and pattern recognition algorithm to the camera images.

54. A system for localizing a medical tool, the system comprising:
a medical tool for use in a medical procedure with a patient;
a localization system associated with the medical tool that locates the medical tool in a three-dimensional coordinate system, the localization system comprising a reference target having a fixed and known position in the coordinate system, the reference target being remote from the medical tool; and
a computer in communication with the localization system, the computer being programmed to (1) receive data from the localization system, and (2) determine the position of the medical tool in the coordinate system at least partially on the basis of data received from the localization system.

55. The system of claim 54 wherein the localization system further comprises a sensor attached to the medical tool at a known position with respect to a point of interest on the medical tool, the sensor being configured to sense the reference target and generate data indicative of the reference target's position in the coordinate system, wherein the data received by the computer from the localization system comprises the sensor data from the sensor, and wherein the computer is programmed to determine the position of the medical tool in the coordinate system at least partially on the basis of the sensor data.

56. The system of claim 55 wherein the sensor is a camera having a field of view within which the reference target at least partially resides, wherein the sensor data comprises image data generated by the camera of at least a portion of the reference target, and wherein the computer is programmed to determine the position of the medical tool in the coordinate system at least partially on the basis of the image data from the camera.

57. The system of claim 56 wherein the reference target comprises a plurality of identifiable marks thereon disposed in a known spatial relationship with each other.

58. The system of claim 57 wherein the identifiable marks comprise a plurality of passive reflectors.

59. The system of claim 58 wherein the identifiable marks comprise a plurality of printed marks visible to the camera.

60. The system of claim 59 wherein the printed marks are arranged in a grid pattern.

61. The system of claim 57 wherein the computer is further programmed to determine, in substantially real-time, the position of the medical tool in the coordinate system at least partially on the basis of the image data from the camera.

62. The system of claim 57 wherein the image data of the reference target generated by the camera comprises image data of at least a portion of the reference target.

63. The system of claim 58 wherein the medical tool is a medical imaging device.

64. The system of claim 63 wherein the medical imaging device is a freehand ultrasound probe.

65. The system of claim 57 wherein the medical tool is a surgical instrument.

66. The system of claim 57 wherein the medical tool is a bite block for use in treating head or neck lesions.

67. A system for localizing a medical imaging probe, the system comprising:
a reference target having a known position in a fixed coordinate system;
a medical imaging probe for receiving data from which a medical image of a patient is generated, the probe being remote from the reference target;
a tracking camera attached to the probe for tracking the reference target and generating at least one image within which the reference target is depicted; and
a computer configured to (a) receive the camera image and (b) process the received camera image to determine the position of the device relative to the coordinate system in 3D coordinate space.
